(19) United States
(12) Patent Application Publication
(10) Pub. No.: US 2024/0358897 A1
(43) Pub. Date: Oct. 31, 2024

(54) ANTIMICROBIAL CATHETER COATINGS WITH PASSIVE ANTIFUNGAL PROPERTIES

(71) Applicant: Becton, Dickinson and Company, Franklin Lakes, NJ (US)

(72) Inventors: David Colton Jackson, Cottonwood Heights, UT (US); James Freasier, Salt Lake City, UT (US); Ejaz Sajedul Haque, Salt Lake City, UT (US)

(21) Appl. No.: 18/140,510

(22) Filed: Apr. 27, 2023

(51) Int. Cl. A61L 29/08

(52) U.S. Cl. CPC A61L 29/085; A61L 2300/104; A61L 2300/25; A61L 2300/252; A61L 2300/404; A61L 2300/442; A61L 2300/606; A61L 2420/02; A61L 2420/04

(57) ABSTRACT

A medical device having resistance to Candida albicans yeast and antimicrobial activity includes a medical device surface having a copolymer coating of antifungal moieties and ionic binding moieties. The copolymer coating is obtained by copolymerizing antifungal monomers selected from tricyclodecane dimethanol diacrylate, 4-tert-butylcyclohexyl acrylate, neopentyl glycol diacrylate, trimethylcyclohexyl methacrylate, isobornyl acrylate, and mixtures thereof and ionic binding monomers selected from acrylic acid, methyl acrylate, and mixtures thereof. The coating may be loaded with elutable antimicrobial substances ionically bound to the ionic binding moieties. The antimicrobial substances may be cationic, such as, a cationic dye such as ethyl violet chloride, a cationic drug such as chlorhexidine diacetate, cationic peptides such as cecropins, magainins, protegrins, and bactenicins, or ionic silver. The medical device may be a catheter having an intraluminal surface and an extraluminal surface, and the copolymer coating is disposed on the intraluminal surface and the extraluminal surface.

### BACKGROUND

The present disclosure relates to antifungal coatings for medical devices. The disclosure further relates to medical device coatings which have antimicrobial and passive antifungal properties. Covalently bound coatings include moieties having passive resistance to the yeast strain, Candida albicans. The coatings are combined with ionically bound active pharmaceutical ingredients (APIs) for elution. The APIs may be selected to provide antimicrobial activity against gram-positive and gram-negative bacteria.

The medical device may include catheters having intraluminal and extraluminal surface coatings with antifungal and antimicrobial properties.

The medical devices disclosed herein include medical devices which are susceptible to bacterial and fungal colonization and infection due to prolonged contact with blood or body fluids. Examples of medical devices include, but are not limited to, catheters, such as central venous catheters (CVCs) and peripherally inserted central catheters (PICCs), urinary catheters, dialysis catheters, as well as auxiliary equipment and tubing that contacts blood, such as blood infusion equipment, plasma collection equipment, dialysis equipment, etc.

Catheters are life saving devices that have become a standard of care. Catheter-related bloodstream infection (CRBSI) and central line-associated bloodstream infection (CLABSI) are caused by the colonization of microorganisms in patients with intravascular catheters and access devices. These infections are an important cause of illness and excess medical costs, as approximately 250,000-400,000 cases of central venous catheter (CVC) associated bloodstream infections occur annually in US hospitals. In addition to the monetary costs, these infections are associated with anywhere from 20,000 to 100,000 deaths each year. Despite guidelines to help reduce healthcare associated infections (HAIs), catheter-related bloodstream infections continue to plague our healthcare system.

Multiple approaches are utilized to mitigate the occurrence of these infections-namely proper insertion site cleaning, good catheter placement practice, and use of antimicrobial agents in or on the catheter tubing to suppress microbial growth.

A majority of the commercially available catheters that are used today do not have any antimicrobial or antifungal action.

To provide antimicrobial and antifungal properties, active agents need to be immobilized into the matrix or coated onto the catheter surface. These catheters, however, have given less than satisfactory results.

Accordingly, there is a need in the art for medical devices, such as catheters, having antifungal and antimicrobial properties.

The subject matter claimed herein is not limited to embodiments that solve any disadvantages or that operate only in environments such as those described above. Rather, this background is only provided to illustrate one example technology area where some implementations described herein may be practiced.

### BRIEF DESCRIPTION OF THE SEVERAL VIEWS OF THE DRAWINGS

Example embodiments will be described and explained with additional specificity and detail through the use of the accompanying drawings in which:

FIG. 1 is a table of five acrylate and diacrylate monomers M1-M5 having passive resistance to Candida albicans yeast strain.

FIG. 2 discloses a mechanism of UV light-induced radical formation with a benzophenone photoinitiator.

FIG. 3 is a flowchart outlining the procedure used to test the surface coatings prepared from the M1-M5 antifungal monomers.

FIG. 4 shows layouts of assay plates utilized for MBEC studies with monomers tested.

FIG. 5 is a graph of the MBEC study results showing Log reductions relative to growth controls for various surface coatings produced from various combinations of monomers M1-M5

### DESCRIPTION OF EMBODIMENTS

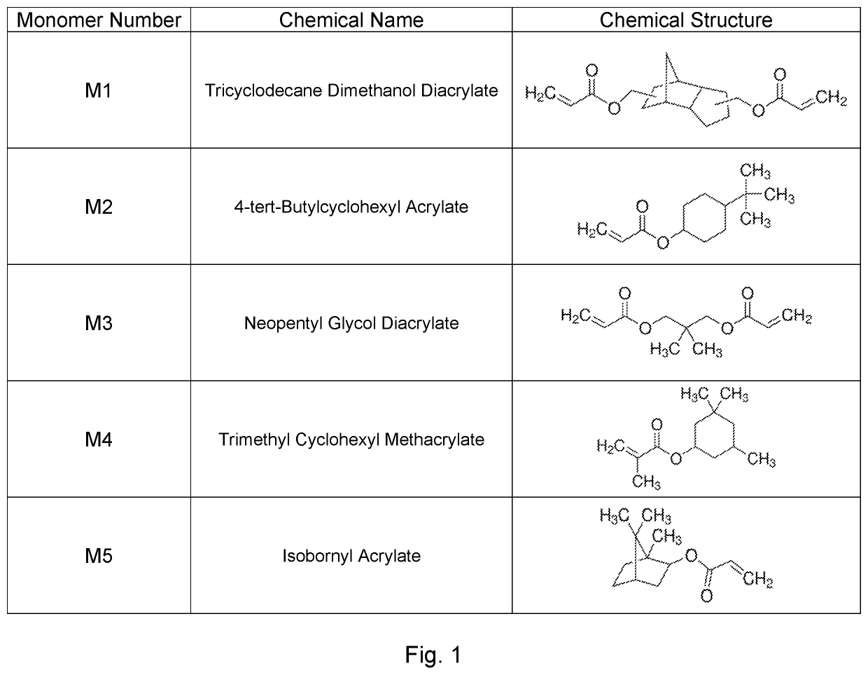

The disclosure relates to a process for preparing antifungal coatings on medical devices using a UV-induced free radical polymerization process. The process may be used to prepare covalently bound intraluminal and extraluminal coatings on catheters. Coatings incorporating antifungal monomers with moieties M1 to M5, shown in FIG. 1, have excellent passive resistance to clinically relevant Candida albicans yeast, such as strain ATCC 90027. The antifungal monomers may be combined with one or more ionic binding eluting monomers to enable active pharmaceutical ingredients (APIs) to be ionically bound to the surface for elution. APIs disclosed herein possess antimicrobial activity and kill of gram-positive Staphylococcus aureus, such as strain ATCC 33592, and gram-negative Pseudomonas aeruginosa, such as strain ATCC 27853.

The process for forming the covalently bound coatings uses a photoinitiator capable of generating radical species upon exposure to UV light to covalently bond ratios of Candida resistant copolymers to the medical device surface. A copolymer graft containing ionic binding monomers can be subsequently loaded with an antimicrobial cationic substance, including but not limited to, a cationic dye such as methylene blue and ethyl violet chloride, a cationic drug such as chlorhexidine diacetate, cationic peptides such as cecropins, magainins, protegrins, and bactenicins, or ionic silver.

U.S. Pat. No. 8,920,886 discloses catheter coatings which focus on the use of carboxylic acid containing acrylate groups (e.g., acrylic acid (CAS #79-10-7) and methyl acrylate (CAS #96-33-3)). In contrast, the antifungal coatings for medical devices disclosed herein replace all or a portion of conventional acrylate monomers with acrylate and diacrylate monomers comprising cyclic hydrocarbons, esters, tertiary butyl moieties, and non-aromatic hydrocarbon moieties which exhibit resistance to Candida albicans. Non-limiting examples of antifungal monomers that show passive resistance to Candida albicans include M1: tricyclodecane dimethanol diacrylate (CAS #43048-08-4), M2: 4-tert-butylcyclohexyl acrylate (CAS #84100-23-2), M3: neopentyl glycol diacrylate (Cas #2223-82-7), M4: trimethyl cyclohexyl methacrylate (CAS #7779-31-9), and M5: isobornyl acrylate (CAS #5888-33-5), shown in FIG. 1. Polymer coatings prepared from these monomers, including mixtures of these monomers, possess passive antifungal activity. The polymer coatings can be loaded with one or more elutable antimicrobial substances to provide antimicrobial activity.

The disclosed antifungal coatings for medical devices utilize radical polymerization to graft copolymer monomer agents to the catheter surface. Surface graft polymerization using ultraviolet (UV) light is an efficient and convenient method for modifying polymer surfaces. One common strategy for surface graft polymerization uses a photoinitiator which produces radicals when exposed to UV light. These then react with monomers to initiate polymer chain growth. More specifically, the photoinitiator generate radical species upon exposure to UV light which abstract hydrogen atoms from the polymer surface, thereby creating surface-bound radicals capable of initiating graft polymerization of monomers in solution. Non-limiting examples of a photoinitiator includes benzophenone (BP), 2,2-dimethoxy-2-phenylacetophenone (DPA), and related molecules.

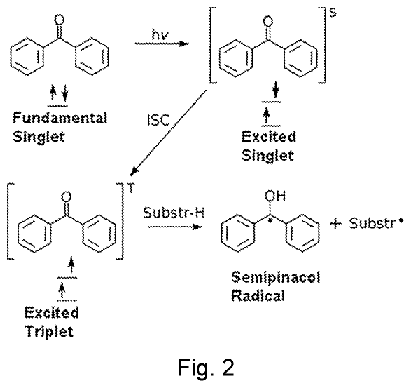

The mechanism of UV light-induced radical formation with a benzophenone photoinitiator in this process is shown in FIG. 2. Gutiérrez-Villarreal, M. H, and J. G. Guzmán-Moreno. “Surface graft polymerization of N-vinylcaprolactam onto polylactic acid film by UV irradiation.” Journal of Polymer Research 20.6 (2013): 1-6. The photoinitiator enters a free radicalized state following exposure to a spectrum of UV-A to UV-C radiation, about 100 nm to 400 nm. The excited electron then undergoes intersystem crossing and enters a highly reactive triplet state, generating a diradical. It then abstracts a hydrogen atom from a surrounding molecule, either a graft monomer or co-initiator. This radical propagates to surrounding monomers in solution to produce a covalently bonded coating at the medical device surface.

The disclosed surface graft polymerization reaction may be performed in a grafting reactor which holds a polymerizable aqueous solution of monomers, which polymerize to form the polymer or copolymer coating grafted onto the surface of a medical device. The monomer solution comprises an antifungal monomer selected from tricyclodecane dimethanol diacrylate, 4-tert-butylcyclohexyl acrylate, neopentyl glycol diacrylate, trimethylcyclohexyl methacrylate, isobornyl acrylate, and mixtures thereof. In some embodiments, the monomer solution comprises two or more antifungal monomers.

In some embodiments, the monomer solution further comprises an ionic binding monomer. In some embodiments, the ionic binding monomer is selected from acrylic acid, methyl acrylate, and mixtures thereof.

The grafting reactor may comprise UV light transmissible walls made of glass or a material that can transmit UV light. UV light assemblies disposed outside the grafting reactor shine UV light through the walls and into the interior of the grafting reactor. The UV light may range from about 100 nm to about 400 nm. This includes UVA (315-400 nm), UVB (280-315 nm), and UVC (100-280 nm). In this way, the medical device, such as catheters, may be dipped into the polymerizable solution and exposed to UV light simultaneously to cause the polymerization reaction and form the antifungal coating.

A copolymer graft containing ionic binding monomers may be exposed to a solution containing one or more antimicrobial substances selected to provide antimicrobial activity against gram-positive and gram-negative bacteria, including, but not limited to, gram-positive Staphylococcus aureus and gram-negative Pseudomonas aeruginosa. In some embodiments, the antimicrobial substances are cationic, including but not limited to, a cationic dye such as methylene blue and ethyl violet chloride, a cationic drug such as chlorhexidine diacetate, cationic peptides such as cecropins, magainins, protegrins, and bactenicins, or ionic silver.

Antifungal coatings for medical devices are produced via photochemical reactions catalyzed by UV radiation. In some embodiments, the medical device is a catheter having intraluminal and extraluminal surfaces. Free radical polymerization may be used to graft copolymers from the intraluminal and extraluminal surfaces of the catheter simultaneously. The graft copolymers may be prepared from a mixture of monomers that have a ratio of monomers containing passive and active eluting moieties. These copolymers are covalently bound to the catheter. They can then be loaded with cationic active pharmaceutical ingredients (APIs) for elution. The APIs may be selected to provide antimicrobial activity against gram-positive and gram-negative bacteria. This process expands on the prior art of catheter coatings by disclosing coatings made with a unique set of monomers resistant to Candida albicans and which may be loaded with one or more antimicrobial agents to provide broad spectrum of antimicrobial and antifungal activity.

Thus, disclosed catheter coatings exhibit passive resistance to Candida albicans in addition to controlled release of antimicrobial substances for active antimicrobial effect against gram-positive Staphylococcus aureus and gram-negative Pseudomonas aeruginosa. The same principles can be adapted to other catheter materials with unique physical and chemical properties.

Other features and advantages of the disclosed invention are apparent from the example that follows. The example illustrates different aspects and embodiments of the present invention and how to make and practice them. The example does not limit the claimed invention. Although methods and materials similar or equivalent to those described herein can be used in the practice of the invention, suitable methods and materials are described below. Based on the present disclosure the skilled artisan can identify and employ other components and methodology useful for practicing the present invention.

### Example 1

Surface coatings produced from five acrylate and diacrylate monomers (M1-M5; FIG. 1) were prepared and confirmed to exhibit passive resistance properties to Candida albicans utilizing standard MBEC (Minimum Biofilm Eradication Concentration) Assay procedures. The photoinitiator 2,2-dimethoxy-2-phenylacetophenone (DPA), having the chemical structure shown below, was used to prepare the surface coatings.

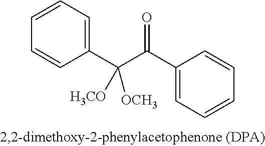 [SMILES: COC(OC)(C(=O)c1ccccc1)c1ccccc1]

The MBEC Assay is a high throughput screening assay used to determine the efficacy of antimicrobials against biofilms of a variety of microorganisms. The MBEC Assay Biofilm Inoculators consist of a plastic lid with 96 pegs a corresponding receiver plate.

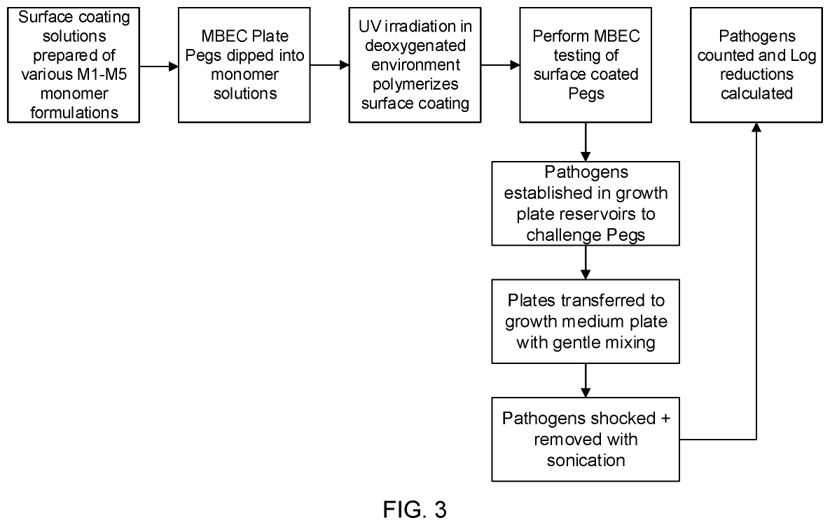

To assess the monomer's effectiveness against clinically relevant strains, surface coatings were polymerized onto the pegs of the assay devices and shipped out for MBEC testing. This allowed pathogens to be established on the pegs under batch conditions with gentle mixing, and then moved to another receiver plate with various reactor conditions of choice to test biofilm or yeast viability. The study tested the resistance to adhesion and Candida albicans formation. To do this, surface coated pegs were cultured in their respective pathogen, transferred to a receiving plate and sonicated, additional serial dilutions and plating were performed, and then pathogen colonies were counted, and log reductions calculated. FIG. 3 is a flowchart outlining the procedure used to test the surface coatings prepared from the M1-M5 antifungal monomers.

Surface coatings were prepared on the pegs utilizing a total solution concentration of 50/50 v %/v % monomer mix/dioxolane ratio and 5% w %/v % photoinitiator 2,2-dimethoxy-2-phenylacetophenone. 200 μL of each polymerization solution was prepared and added to a corresponding peg receiver reservoir. Pegs were soaked in this solution and put in a UV reactor oven fed with a nitrogen blanket. When measured oxygen content within the oven was less than 200 ppm, the samples underwent a UV cure from UVA bulbs for ten minutes.

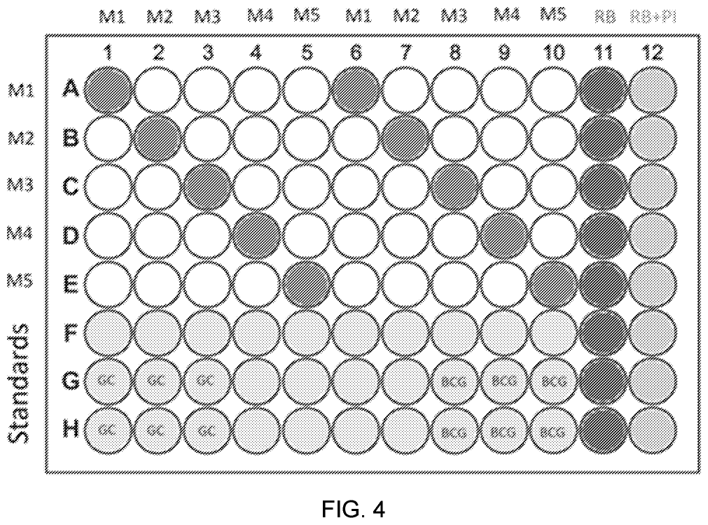

Referring to FIG. 4, surface coating solutions were prepared so that the pegs for rows A-E and columns 1-5 were surface coated by 50/50 ratios of the acrylate monomers M1-M5, and repeated in columns 6-10. That is, Rows A-E corresponded to surface coatings utilizing a monomer from monomers M1-M5, and Columns 1-5 and 6-10, corresponded to a monomer from monomers M1-M5. Therefore, each peg was prepared to have a surface coating including a mixture of two monomers except the diagonal peg locations in FIG. 4 (A1, B2, C3, D4, E5, A6, B7, C8, D9, and E10) which were prepared from a solution of a single monomer. This allowed for surface coatings of each monomer to be tested twice (MX/MX, represented by the diagonal peg locations) as well as four replicates of 50/50 ratio monomer solution pair variations (MX/MY). Pegs in column 11 were reserved to be dipped/conditioned in reagent blank testing controls of the solvent system dioxolane, column 12 was reserved for the reagent blank+% 5 photo initiator controls, and rows F-H were left blank to be reserved for MBEC procedural pathogen growth control (GC) standards and yeast growth checks (BGC).

An illustration of a plate is provided in FIG. 4, below. FIG. 4 shows layouts of assay plates utilized for MBEC studies with monomers tested. Various ratios and placement of monomers used in surface coating solutions utilized described in the section above.

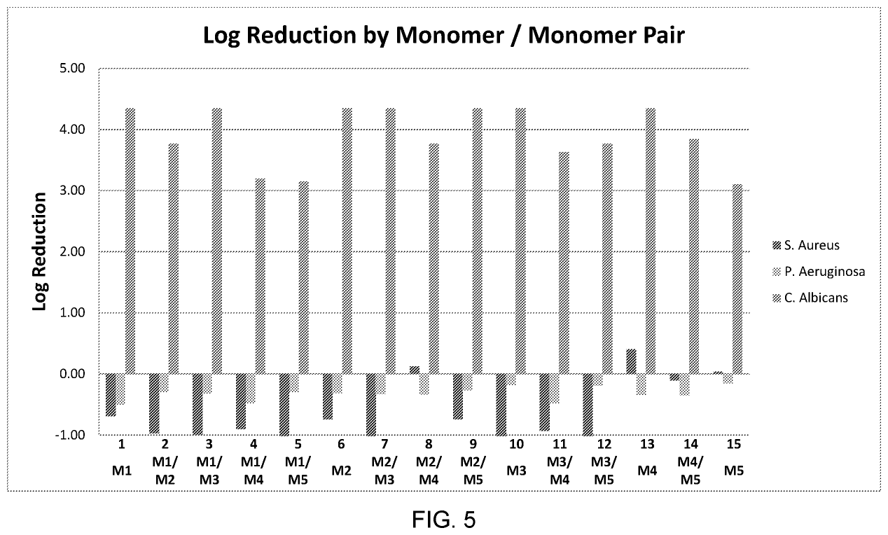

Results of the MBEC studies show several variations of surface coatings prepared from single monomer and combinations of M1-M5 which produced 4-Log reductions (99.99% reduction) in Candida Albicans formation relative to the growth control recovery data as can be seen in in FIG. 5. FIG. 5 is a graph of the MBEC study results showing Log reductions relative to growth controls for various surface coatings produced from various combinations of monomers M1-M5. Several variations of surface coatings utilizing these novel monomers produced satisfactory 4-Log reduction and show a general pathogen resistance to clinically relevant Candida albicans. Surface coatings generated from M1-M5 exhibit a general resistance 3-Log reductions (99.9% reduction) to Candida albicans.

### EMBODIMENTS

Various embodiments are listed below. It will be understood that the embodiments listed below may be combined with all aspects and other embodiments in accordance with the scope of the invention.
Embodiment 1. A method for preparing an antifungal coating on a medical device comprising: obtaining a medical device comprising a medical device surface, wherein the medical device surface comprises a photoinitiator which generates radicals upon exposure to UV light; exposing the medical device surface to a monomer solution comprising an antifungal monomer; and irradiating the photoinitiator with UV light to cause radical polymerization and graft the antifungal monomer to the medical device surface and form the antifungal coating.
Embodiment 2. The method of Embodiment 1, wherein the antifungal monomer is selected from tricyclodecane dimethanol diacrylate, 4-tert-butylcyclohexyl acrylate, neopentyl glycol diacrylate, trimethylcyclohexyl methacrylate, isobornyl acrylate, and mixtures thereof.
Embodiment 3. The method of Embodiment 2, wherein the monomer solution comprises two or more antifungal monomers.
Embodiment 4. The method of any preceding Embodiment, wherein the medical device comprises a catheter having an intraluminal surface and an extraluminal surface, and wherein the medical device surface comprises the intraluminal surface and the extraluminal surface.
Embodiment 5. The method of any preceding Embodiment, wherein the monomer solution further comprises an ionic binding monomer and wherein the step of irradiating the photoinitiator with UV light causes radical polymerization of the antifungal monomer and ionic binding monomer to form a copolymer coating with antifungal and ionic binding moieties.
Embodiment 6. The method of Embodiment 5, wherein the ionic binding monomer is selected from acrylic acid, methyl acrylate, and mixtures thereof.
Embodiment 7. The method of Embodiments 5 or 6, further comprising exposing the copolymer coating to an antimicrobial substance which binds to the ionic binding moieties.
Embodiment 8. The method of Embodiment 7, wherein the antimicrobial substance is a cationic antimicrobial dye.
Embodiment 9. The method of Embodiment 8, wherein the cationic antimicrobial dye is selected from methylene blue and ethyl violet.
Embodiment 10. The method of Embodiment 7, wherein the antimicrobial substance is chlorhexidine diacetate.
Embodiment 11. The method of Embodiment 7, wherein the antimicrobial substance is ionic silver.
Embodiment 12. The method of Embodiment 7, wherein the antimicrobial substance is a cationic peptide selected from cecropins, magainins, protegrins, and bactenicins.
Embodiment 13. The method of any preceding Embodiment, wherein the photoinitiator is a radical generating species selected from 2,2-dimethoxy-2-phenylacetophenone, benzophenone, or related species, and mixtures thereof.
Embodiment 14. A medical device having antifungal and antimicrobial activity comprising: a medical device surface having a copolymer coating comprising antifungal moieties and ionic binding moieties, wherein the copolymer coating is obtained by copolymerizing antifungal monomers selected from tricyclodecane dimethanol diacrylate, 4-tert-butylcyclohexyl acrylate, neopentyl glycol diacrylate, trimethylcyclohexyl methacrylate, isobornyl acrylate, and mixtures thereof and ionic binding monomers selected from acrylic acid, methyl acrylate, and mixtures thereof; and an antimicrobial substance ionically bound to the ionic binding moieties.
Embodiment 15. The medical device of Embodiment 14, wherein the medical device comprises a catheter having an intraluminal surface and an extraluminal surface, and wherein the copolymer coating is disposed on the intraluminal surface and the extraluminal surface.
Embodiment 16. The medical device of Embodiments 14 or 15, wherein the antimicrobial substance is a cationic antimicrobial dye.
Embodiment 17. The medical device of Embodiment 16, wherein the cationic antimicrobial dye is selected from methylene blue and ethyl violet.
Embodiment 18. The medical device of Embodiments 14 or 15, wherein the antimicrobial substance is chlorhexidine diacetate.
Embodiment 19. The medical device of Embodiments 14 or 15, wherein the antimicrobial substance is ionic silver.
Embodiment 20. The medical device of Embodiments 14 or 15, wherein the antimicrobial substance is a cationic peptide selected from cecropins, magainins, protegrins, and bactenicins.
Embodiment 21. The medical device of Embodiments 14 or 15, wherein the antimicrobial substance is selected from a cationic antimicrobial dye, chlorhexidine diacetate, ionic silver, a cationic peptide, and combinations thereof.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention includes modifications and variations that are within the scope of the appended claims and their equivalents.

1. A method for preparing an antifungal coating on a medical device comprising:
obtaining a medical device comprising a medical device surface, wherein the medical device surface comprises a photoinitiator;
exposing the medical device surface to a monomer solution comprising an antifungal monomer; and
irradiating the photoinitiator with UV light to cause radical polymerization and graft the antifungal monomer to the medical device surface and form the antifungal coating.

2. The method of claim 1, wherein the antifungal monomer is selected from tricyclodecane dimethanol diacrylate, 4-tert-butylcyclohexyl acrylate, neopentyl glycol diacrylate, trimethylcyclohexyl methacrylate, isobornyl acrylate, and mixtures thereof.

3. The method of claim 2, wherein the monomer solution comprises two or more antifungal monomers.

4. The method of claim 1, wherein the medical device comprises a catheter having an intraluminal surface and an extraluminal surface, and wherein the medical device surface comprises the intraluminal surface and the extraluminal surface.

5. The method of claim 1, wherein the monomer solution further comprises an ionic binding monomer and wherein the step of irradiating the photoinitiator with UV light causes radical polymerization of the antifungal monomer and ionic binding monomer to form a copolymer coating with antifungal and ionic binding moieties.

6. The method of claim 5, wherein the ionic binding monomer is selected from acrylic acid, methyl acrylate, and mixtures thereof.

7. The method of claim 5, further comprising exposing the copolymer coating to an antimicrobial substance which binds to the ionic binding moieties.

8. The method of claim 7, wherein the antimicrobial substance is a cationic antimicrobial dye.

9. The method of claim 8, wherein the cationic antimicrobial dye is selected from methylene blue and ethyl violet.

10. The method of claim 7, wherein the antimicrobial substance is chlorhexidine diacetate.

11. The method of claim 7, wherein the antimicrobial substance is ionic silver.

12. The method of claim 7, wherein the antimicrobial substance is a cationic peptide selected from cecropins, magainins, protegrins, and bactenicins.

13. The method of claim 1, wherein the photoinitiator is a radical generating species selected from 2,2-dimethoxy-2-phenylacetophenone, benzophenone, and mixtures thereof.

14. A medical device having antifungal and antimicrobial activity comprising:
a medical device surface having a copolymer coating comprising antifungal moieties and ionic binding moieties, wherein the copolymer coating is obtained by copolymerizing antifungal monomers selected from tricyclodecane dimethanol diacrylate, 4-tert-butylcyclohexyl acrylate, neopentyl glycol diacrylate, trimethylcyclohexyl methacrylate, isobornyl acrylate, and mixtures thereof and ionic binding monomers selected from acrylic acid, methyl acrylate, and mixtures thereof; and
an antimicrobial substance ionically bound to the ionic binding moieties.

15. The medical device of claim 14, wherein the medical device comprises a catheter having an intraluminal surface and an extraluminal surface, and wherein the copolymer coating is disposed on the intraluminal surface and the extraluminal surface.

16. The medical device of claim 14, wherein the antimicrobial substance is a cationic antimicrobial dye.

17. The medical device of claim 16, wherein the cationic antimicrobial dye is selected from methylene blue and ethyl violet.

18. The medical device of claim 14, wherein the antimicrobial substance is chlorhexidine diacetate.

19. The medical device of claim 14, wherein the antimicrobial substance is ionic silver.

20. The medical device of claim 14, wherein the antimicrobial substance is a cationic peptide selected from cecropins, magainins, protegrins, and bactenicins.
